(19) United States
(12) Patent Application Publication
(10) Pub. No.: US 2004/0132827 A1
(43) Pub. Date: Jul. 8, 2004

(54) Phenylephrine tannate, pyrilamine tannate and dextromethorphan tannate salts in pharmaceutical compositions

(75) Inventors: Jeffrey S. Kiel, Gainesville, GA (US); H. Greg Thomas, Villa Rica, GA (US); Narasimhan Mani, Gainesville, GA (US)

Correspondence Address: WOOD, HERRON & EVANS, LLP, 2700 CAREW TOWER, 441 VINE STREET, CINCINNATI, OH, 45202, US

(73) Assignee: PediaMed Pharmaceuticals, Inc., Exton, PA

(21) Appl. No.: 10/645,977

(22) Filed: Aug. 22, 2003

Related U.S. Application Data
(63) Continuation-in-part of application No. 10/047,578, filed on Oct. 26, 2001.

(51) Int. Cl. A61K 31/137

(52) U.S. Cl. 514/649

(57) ABSTRACT

Compositions of tannate salts of phenylephrine, pyrilamine, and dextromethorphan produced by a method that allows for the in-situ conversion and incorporation of the tannate salts in a single dosage form. The conversion process includes dissolving salts of phenylephrine, pyrilamine, and dextromethorphan in a solvent and mixing with a dispersing agent and tannic acid to generate tannate salts. The tannate salts may be further processed without further purification or isolation to single dosage forms, such as tablets and suspension.

### CROSS-REFERENCE TO RELATED APPLICATIONS

This application is a continuation-in-part of U.S. patent application Ser. No. 10/047,578, filed Oct. 26, 2001, by Jeffrey S. Kiel et al., which is hereby incorporated by reference herein in its entirety.

### FIELD OF INVENTION

The present invention relates generally to the field of tannate chemistry and more specifically to methods for processing phenylephrine tannate, pyrilamine tannate, and dextromethorphan tannate compositions for use in the treatment of coryza and the compositions produced.

### BACKGROUND OF THE INVENTION

Pyrilamine, phenylephrine, and dextromethorphan are well known, both in their free base form as well as salts, such as hydrochloride, citrate, maleate, tannate, etc. These compounds, when in the form of tannate salts, are particularly desirable due to their stability. As a result, they may be combined without any untoward side effects. The tannate salts have also been found to have better organoleptic properties such as taste, in comparison to other salts or free base forms of such compounds. In addition, tannate salts are relatively large molecules, which results in absorption over prolonged intervals of time. This reduces the frequency of administration of the compounds and thereby improves patient compliance factors. Due to the above properties, such compounds are amenable to use as active pharmaceutical ingredients in a composition.

Phenylephrine, known chemically as L-m-hydroxy.alpha.[(methylamino)methyl]benzyl alcohol, is a synthetic, optically active sympathomimetic compound, which has a 2′-amine functional group in its molecular structure. Phenylephrine hydrochloride is available as a white, odorless, non-hygroscopic, crystalline compound, in the form of the levorotory isomer possessing a bitter taste. It is freely soluble in water and has a melting point of about 143° C.

Pyrilamine, one of the oldest and most enduring antihistaminic compounds, known chemically as N-[(4-methoxyphenyl)methyl]-N′,N′-dimethyl-N-2-pyridinyl-1,2-ethanediamine, has a 3°-amine functional group present in its molecular structure and is an oily liquid. Pyrilamine hydrochloride is freely soluble in water, whereas the maleate salt is slightly soluble in water and has a melting point of about 101° C.

Dextromethorphan (C₁₈H₂₅NO) is a well known antitussive, known chemically as d-3-methoxy-N-methylmorphinan, also has a 3°-amine functional group present in its molecular structure. The hydrobromide salt occurs as the monohydrate, and has a melting point of 122-124° C.

Tannic acid, also known as tannin, is a well-known naturally occurring substance typically produced from Turkish or Chinese nutgall. Chemically, these acids are described as polymers of different hydroxysodium benzoates. The chemistry of the tannins is complex and non-uniform. As a result the tannic acid used to produce antihistamine and decongestant tannate salts is variable in its purity. The water content of tannic acid varies from 5-10% and the molecular weight is about 1700.

Pyrilamine, phenylephrine, and dextromethorphan in the form of their tannate salts are typically prepared by reacting the free bases of phenylephrine, pyrilamine, or dextromethorphan, with tannic acid in the presence of a volatile solvent, usually isopropanol or water. The reaction mixture is stirred for about 1 hour while maintaining the mixture at 60-70° C. The reaction mixture is subsequently cooled to room temperature and then filtered, washed and vacuum dried to obtain the tannate salts. The yield of the tannate salt products using such methods typically varies from about 70% when using the isopropanol route to 90-97% using the water method. The purity of the tannate salts produced as described above is variable. The purity ranges form 85-90% when using the isopropanol route to about 90-98% when using the water route.

Due to the large nature of the tannate molecule, the percentage of antihistamine or decongestant or antitussive free base within the tannate salt is significantly lower than that in other salt forms such as the hydrochloride or maleate. The presence of low active percentages of antihistamine, decongestant, or antitussive and the variable purity of the commercially available antihistamine, decongestant, and antitussive tannate salts result in the stoichiometry of the active free base to tannic acid in the tannate salts being different from batch to batch. This may result in significant dosing and processing problems during manufacture and increase the likelihood that commercially available pharmaceutical compositions contain variable, and in some instances, sub-therapeutic levels of active pharmaceutical ingredients.

Therefore, it would be desirable if pharmaceutical compositions containing pyrilamine, phenylephrine, and dextromethorphan tannates could be prepared with reduced variability in active drug content and increased certainty that the active pharmaceutical ingredients are delivered within a therapeutic range.

### SUMMARY OF THE INVENTION

In accordance with the present invention and the contemplated problems which have and continue to exist in this field, the present invention provides a manufacturing method for in-situ conversion and incorporation of tannate salts of pyrilamine, phenylephrine, and dextromethorphan in a single dosage form. The present invention also provides for pharmaceutical compositions including these tannate salts. These single dosage forms may include suspensions and tablets.

The present invention involves addition of a dispersing agent and tannic acid to purified water to which an aqueous solution of the active pharmaceutical ingredient, phenylephrine, pyrilamine, or dextromethorphan, is added slowly to generate a water insoluble tannate salt. The presence of the dispersing agent prevents the clumping and aggregation of the tannate salt formed.

The resulting dispersion of the tannate salt in water may then be further processed by transferring to a suspending medium, whose composition includes thickening agents, sweetening/flavoring agents, anti-caking agents, co-solvents, pH adjusting agents, preservatives, coloring agents, and purified water. The resulting mixture can be processed into suitable liquid dosage forms, such as a suspension containing the tannate salts. In a preferred form, each 5 ml of the suspension contains 30 mg of pyrilamine tannate, 12.5 mg of phenylephrine tannate, and 25 mg of dextromethorphan tannate.

In an alternate method, pyrilamine, phenylephrine, and dextromethorphan salts are dissolved in the water and a wet granulation is prepared by spraying the active ingredient solutions onto a mixture of tannic acid, dispersing agent and diluent. The granulation is subsequently dried and then is dry blended with additional diluent, and with sweetening, hardness-increasing, coloring, and flavoring agents as necessary. The resulting granulate can be processed into tablets. In a preferred form, each 550 mg tablet contains 30 mg of pyrilamine tannate, 25 mg of phenylephrine tannate, and 25 mg of dextromethorphan tannate.

By starting with the commonly available salt or the free base form of the active pharmaceutical ingredient, which is subsequently converted and incorporated in-situ as a tannate salt, the invention provides an efficient, inexpensive, and reproducible method to manufacture products containing tannate salts as active ingredients.

By using the tannate salt of the active pharmaceutical ingredient, the present invention provides a dosage form which affords a release of the active over prolonged intervals of time, and thereby improving patient compliance factors. Since the tannate salt of the active pharmaceutical ingredient is generated and incorporated in-situ into the dosage form during the manufacturing process, the purification and drying steps previously required for the isolation of the tannate salt are eliminated.

### DETAILED DESCRIPTION

The present invention provides a manufacturing method for in-situ conversion and incorporation of tannate salts of pyrilamine, phenylephrine, and dextromethorphan in a single dosage form. The present invention also provides for pharmaceutical compositions including these tannate salts. In one embodiment, the present invention provides a manufacturing process for the in-situ conversion and incorporation of a combination of tannate salts of pyrilamine, phenylephrine, and dextromethorphan into a therapeutic liquid suspension dosage form, and also provides compositions of the same. In another embodiment, the present invention provides a manufacturing process for in-situ conversion and incorporation thereof, of pyrilamine, phenylephrine, and dextromethorphan as tannate salts into suitable solid dosage forms such as tablets and capsules, and also provides compositions of the same.

In general, in a first embodiment, the invention features a manufacturing process for the in-situ conversion and incorporation of a combination of tannate salts of pyrilamine, phenylephrine, and dextromethorphan into a therapeutic suspension liquid dosage form which includes the steps of first dissolving salts of the active pharmaceutical ingredients, pyrilamine, phenylephrine, and dextromethorphan, in a first solvent at a temperature and pH value that will not cause the composition to degrade. This forms a first solution. In one embodiment, these salts are dissolved in purified water to form the first solution. Pyrilamine, phenylephrine, and dextromethorphan may be dissolved separately or together. By dissolving the salts of pyrilamine, phenylephrine, and dextromethorphan in water, the dissociation of the salt into its free base and conjugate acid forms is effected.

Next, a dispersing agent is added to tannic acid in a second solvent, under stirring, to form a first dispersion. In particular, this first dispersion is a solution of a dispersing agent and tannic acid in water. In particular, the first dispersion is formed in this embodiment by adding the dispersing agent, such as magnesium aluminum silicate, to purified water under stirring, and then adding the tannic acid under stirring to form a mixture in which the dispersing agent is uniformly dispersed throughout the solution. In forming the first dispersion, the dispersing agent and tannic acid may be mixed into the purified water by use of a high shear mixer or other apparatus, such as a planetary mixer.

Next, a part or whole of the first solution is transferred to the first dispersion under stirring to form a second solution including the tannate salts of pyrilamine, phenylephrine, and dextromethorphan as a precipitate in water. The first solution may be added to the first dispersion in small portions. In one particular embodiment, the first solution may be added in small portions to the first dispersion while stirring at low speeds to form the second solution. As this occurs, the free base form of the salts react with the tannic acid to form the tannate salts of pyrilamine, phenylephrine, and dextromethorphan. In particular, the conversion of the active pharmaceutical ingredients of pyrilamine, phenylephrine, and dextromethorphan to the tannate salt occurs by the reaction of functional groups, such as secondary amines in the molecular structure of phenylephrine, and tertiary amines in the molecular structure of pyrilamine and dextromethorpan, with the tannic acid. Since the tannate salt formed is larger in size and has low solubility in purified water, it is precipitated out of the second solution, resulting in the tannate salts dispersed in liquid, which may be water. The dispersing agent prevents the clumping and aggregation of the tannate salt generated.

Next, excipients such as thickening, suspending, coloring, sweetening, and flavoring agents are added to water under stirring, to form a third solution. Preservatives, pH adjusting agents, and anti-caking agents are then added to glycerin under stirring to form a second dispersion. Adding the second dispersion in part or as a whole to the third solution under stirring generates a liquid pharmaceutical carrier as a suspension. This liquid pharmaceutical carrier may have a pH range of about 3.5 to about 6.5.

Finally, at least a portion of the second solution is added to the liquid pharmaceutical carrier to produce a liquid dosage form including pharmaceutically active tannate salts, and particularly the tannate salts of pyrilamine, phenylephrine, and dextromethorphan.

The pyrilamine, phenylephrine, and dextromethorphan which may be used as free bases or as salts having anionic functional groups of maleate, citrate, chloride, bromide, acetate, and sulfate. In one embodiment, pyrilamine may be obtained as a maleate salt, phenylephrine may be obtained as a hydrochloride salt, and dextromethorphan may be obtained as a hydrobromide salt. The source of the tannic acid used in the present invention may be natural or synthetic. Exemplary dispersing agents are magnesium aluminum silicate (MAS), xanthan gum and cellulose compounds.

In a particular embodiment, pyrilamine, phenylephrine, and dextromethorphan are each present in the composition in a range of about 0.05% to about 25.0% by weight. The step of forming a first solution by dissolving the salts of the pyrilamine, phenylephrine, and dextromethorphan in water at a maximum temperature that will not cause the composition to degrade is carried out in a temperature range of about 20° C. to about 50° C. As described above, this step of forming a first solution by dissolving the salt of the pyrilamine, phenylephrine, or dextromethorphan in water occurs at a pH value that will not cause the composition to degrade. In particular, this pH may be in the range of about 3 to about 11.

The dispersing agent may be magnesium aluminum silicate (MAS) present in a range of about 0.05% to about 5.0% by weight, and the tannic acid may be present in a range of about 0.01% to about 30.0% by weight. The ratio of magnesium aluminum silicate to tannic acid by weight is in the range of about 0.1:1 to about 100:1. Additionally, the ratio of solid components to water by weight in the first dispersion is in the range of about 1:25. Additionally, the ratio of tannic acid to the active pharmaceutical ingredients by weight is in the ratio of about 2:1 to about 10:1.

This embodiment of the present invention involves further processing of the tannate salts into a liquid dosage form. As described above, thickening, suspending, coloring, sweetening and flavoring agents are added to water under stirring to form a third solution. In a particular embodiment, the thickening agent may be magnesium aluminum silicate present in a range of about 0.5% to about 10.0% by weight; the suspending agent may be xanthan gum present in a range of about 0.5% to about 10.0% by weight; the sweetening agents may be sucrose present in a range of about 5.0% to about 50.0% and sucralose and magnasweet MM-100 may each be present in a range of about 0.01% to about 3.0% by weight; the flavoring agent may be artificial grape and is present in a range of about 0.01% to about 2.0% by weight; and the solvent may be water and is present in a range of about 10.0% to about 85.0% by weight.

Additionally, in this embodiment, preservatives, pH adjusting, and anti-caking agents may be added to glycerin under stirring to form the second dispersion. In this embodiment, the preservative used may be methylparaben present in the range of about 0.01% to about 1% by weight; the pH adjusting agents may be sodium benzoate, citric acid, and sodium citrate, each present in an amount in a range of about 0.05% to about 1% by weight; the anti-caking agent may be MAS in the range of about 0.5% to about 10% by weight; and the dispersion medium, glycerin, may be present in the range of about 2.5% to about 20% by weight.

In the method of the present invention, the final pH of the suspension of the liquid dosage form is in the range of about 3.5 to about 6.5. The final product is for immediate or prolonged release of the active ingredients.

The composition of the present invention is prepared for oral administration in the form of a liquid suspension formulated so that each 5 ml of suspension would contain 30 mg pyrilamine tannate, 12.5 mg phenylephrine tannate, and 25 mg dextromethorphan tannate, when prepared by the methods of the present invention previously described. The composition is useful in the treatment of coryza as phenylephrine functions as a decongestant, pyrilamine functions as an antihistamine, and dextromethorphan functions as an antitussive. Table 1 below shows the initial starting ingredients and amounts for a particular embodiment of the invention.

TABLE 1

| Inqredient | % w/v | Wt. (mq/5 mL) |
|---|---|---|
| Pyrilamine Maleate | 0.32 | 16.00 |
| Phenylephrine HCl, USP | 0.10 | 5.00 |
| Tannic Acid, USP | 0.80 | 40.00 |
| Sucrose, NF | 10.00 | 500.00 |
| Glycerin, USP | 7.50 | 375.00 |
| Magnesium Aluminum Silicate, NF | 0.80 | 40.00 |
| Xanthan Gum | 0.45 | 22.50 |
| Sodium Citrate Dihydrate | 1.00 | 50.00 |
| Methylparaben, NF | 0.20 | 10.00 |
| Sodium Benzoate, USP | 0.10 | 5.00 |
| FD&C Red #40 | 0.015 | 0.75 |
| FD&C Blue No. 1 | 0.004 | 0.20 |
| Grape Flavor | 1.30 | 65.00 |
| Dextromethorphan HBr | 0.30 | 15.00 |
| Citric Acid | 0.40 | 20.00 |
| Magnasweet MM-100 | 0.30 | 15.00 |
| Sucralose | 0.20 | 10.00 |
| Purified Water, USP | qs to volume | qs to volume |
| Total: | 100.00 | 850 L |

As noted in Table 1, the excipients used in this embodiment of the formulation are sucrose, sucralose, magnasweet MM-100 and artificial grape flavor as flavoring agents; xanthan gum and magnesium aluminum silicate (MAS) as thickening and anti-caking agents; glycerin as a co-solvent; sodium citrate, citric acid and sodium benzoate as pH adjusting and buffering agents; methylparaben as a preservative; FD&C Red No. 40 and FD&C Blue No. 1 as coloring agents; and purified water.

In this embodiment of the composition, the thickening agents xanthan gum and MAS, the flavoring agents sucrose, sucralose, magnasweet MM-100 and artificial grape and the coloring agents FD&C Red No. 40 and FD&C Blue No. 1 are dispersed in purified water to generate the suspending medium of the liquid pharmaceutical carrier. In one particular embodiment, purified water is placed in a mixing tank and stirred. While stirring, the MAS is first added in small portions and mixed until a uniform dispersion of the MAS in water is obtained. Similarly, the xanthan gum is transferred to the mixture. The sucralose, magnasweet MM-100 and the sucrose are then added and dissolved in the mixture. Mixing speed is adjusted to obtain a sufficient vortex to achieve the wetting of the MAS and xanthan gum and to minimize air entrapment. Typical mixing speeds may be between 500 and 1000 rpm.

The coloring agents FD&C Red No. 40 and FD&C Blue No. 1 are dissolved separately in water in a 600 ml beaker and added to the mixture. The artificial grape flavor is then added to the mixture to form the liquid pharmaceutical carrier.

At least a portion of the second solution including tannate salts of the active pharmaceutical ingredients is then added to the liquid pharmaceutical carrier. Mixing is continued until a uniform dispersion of all the ingredients is obtained in the liquid dosage form. In the final formulation of this particular embodiment, pyrilamine tannate is present at 30 mg per 5 ml dose, phenylephrine tannate is present at 12.5 mg per 5 ml dose, and dextromethorphan tannate is present at 25 mg per 5 ml dose.

In general, in another embodiment, the present invention provides a manufacturing process for in-situ conversion and incorporation thereof, of pyrilamine, phenylephrine, and dextromethorphan as tannate salts into suitable solid dosage forms such as tablets and capsules, for human and veterinary use. Since the tannate salt of the active is generated and incorporated in-situ into the dosage form during the manufacturing process, the isolation, purification and drying, routinely performed in the production of the commercially available tannate compounds, is eliminated.

In this embodiment, in general, the present invention features mixing of a dispersing agent, a diluent and tannic acid, as dry powders, to generate a first powder mixture. An aqueous solution of salts of the active pharmaceutical ingredients (API), phenylephrine, pyrilamine, and dextromethorphan may be sprayed on or added slowly to the dispersing agent/tannic acid mixture to generate the tannate salt. The presence of the dispersing agent prevents the clumping and aggregation of the tannate salt formed and promotes uniformity in the first powder mixture. The tannate salt of the API obtained from the above conversion process, may then be mixed with dry binding/matrix forming agents, and may be wet granulated by spraying a solution of a binder. The granulation may be subsequently dried, milled and then may be dry blended with more diluent, sweetening, hardness increasing, coloring, flavoring and flow agents as necessary. The resulting granulate can be processed into tablets, capsules and other solid dosage forms as necessary.

The method of the present invention first involves the conversion of the API to the tannate salt by the reaction of functional groups in the molecular structure of the API, with tannic acid. The amount and ratio of dispersing agent and tannic acid is determined by the molecular configuration and concentration of the API. By starting with a commonly available salt of the API, which is subsequently converted and incorporated in-situ as a tannate salt, the invention provides an efficient method to manufacture solid dosage forms containing tannate salts as active ingredients.

Tannate pharmaceuticals referred to in this embodiment of the invention are solid dosage forms containing active pharmaceutical ingredients as tannate salts. These dosage forms are indicated for relief of nasal congestion and other allergies such as sinusitis, rhinitis and hay fever. The solid dosage forms include tablets (chewable and swallowable), capsules and the like. Owing to the large size of the tannate molecule, the absorption of the API is delayed and thereby the tablet provides a prolonged effect due to the release of the active over prolonged intervals of time. By forming a tannate salt of the API, the present invention also improves taste, which improves patient compliance factors.

As with most pharmaceutical compositions, the compositions formed by the method of the present invention contain inert substances used as a diluent or vehicle for the drug. These excipients, in the present formulation, may be as follows: mannitol as a diluent, magnesium aluminum silicate (MAS) as a dispersing agent, corn starch as a binder, hydroxypropyl methyl cellulose (HPMC E-10) and xanthan gum as additional binding agents, calcium phosphate as a hardness enhancer, talc as a glidant, magnesium stearate as a lubricant, and grape flavor as a flavoring agent.

The first step of the method of the present invention is the conversion of the active pharmaceutical ingredients into tannate salts. As previously mentioned, the tannate salts of the active pharmaceutical ingredients afford a more prolonged effect due to their slow absorption. The simplest way of preparing the tablet is to use the tannate salt of the active pharmaceutical ingredients as raw material. However, the purity of the commercially available tannate compounds is variable. The stoichiometry of the free base to tannic acid in the raw material is different from batch to batch. This causes significant dosing and processing problems during manufacture.

Therefore, in the present manufacturing process, commonly available salts of the API are converted in-situ into the tannate salt and subsequently incorporated into the tablet. In one embodiment, phenylephrine was obtained as a hydrochloride salt, pyrilamine was obtained as a maleate salt, and dextromethorphan was obtained as a hydrobromide salt.

The salt forms of the active ingredients may be dissolved in purified water. In particular, salts of the active pharmaceutical ingredients, pyrilamine, phenylephrine, and dextromethorphan, are dissolved in a first solvent at a temperature and pH value that will not cause the composition to degrade. This forms a first solution. As described above, in one embodiment, these salts are dissolved in purified water to form the first solution. Pyrilamine, phenylephrine, and dextromethorphan may be dissolved separately or together. By dissolving the salts of pyrilamine, phenylephrine, and dextromethorphan in water, the dissociation of the salt into its free base and conjugate acid forms is effected.

Next, a dispersing agnt, diluent, and tannic acid may be mixed to form a first powder mixture. In one particular embodiment, MAS is used as a dispersing agent and mannitol is used as a diluent. Thus, tannic acid, MAS and mannitol are blended as dry powders.

While mixing the blend, at least a portion of the first solution of the active pharmaceutical ingredients is transferred to the first powder mixture. In particular the first solution may be slowly poured onto the first powder mixture. A ten minute mixing time may be allowed after addition of each active pharmaceutical ingredient. This forms a granulate. MAS present in the blend may serve as a solid support to the tannic acid and aids in the dispersion of the tannate salt formed, thereby preventing any lumps that are formed as a result of the conversion process.

The granulate formed in the mixing process described above may be dried at 45° C. to 60° C. and milled. The drying time is significantly reduced from that previously observed and there is a more uniform free flowing powder mass at the end of the drying step. The granulate, now milled, may then be combined with one or more of diluents, dry binding/matrix forming agents, binding solution, coloring agents, sweetening agents, hardness-increasing agents, flavoring agents, and other excipients. Certain of these substances may include, but are not limited to, MAS, calcium phosphate, HPMC E-10, mannitol, xanthan gum, corn starch, talc, magnesium stearate, and compressible sugar (Di-Pac). In one particular embodiment, the granulate may be dry blended with additional DI-PAC, calcium phosphate, talc, and magnesium stearate and may be tableted. The granulate shows very good flow properties and the tablet hardness may be 10-12 kp.

Based on the conversion step and properties such as flow, ease of blending, drying and milling of the granulation the concentration ranges of the excipients may be as follows:

MAS: 0.10-4.50%

Calcium Phosphate: 1.00-3.00%

HPMC E-10: 1.00-3.00%

Mannitol (wet mass): 15.00-50.00%

Xanthan Gum: 1.50-7.50%

Corn starch: 0.50-2.00%

Talc: 0.10-1.00%

Magnesium stearate: 0.25-0.50%

In the case of the chewable tablets, compressible sugar (Di-Pac) alternatively may be used as a diluent to enhance the palatability of the tablet. The diluent may be introduced in the dry blending stage of the formulation. The granulation manufactured using Di-Pac in the diluent shows good flow and tabletability. In addition, batches of the chewable tablets containing grape flavor may be manufactured. However, those of skill in the art will recognize that any flavors may be used. The concentration ranges of the above excipients are as follows:

Di-Pac: 10.00-50.00%

Grape Flavor: 0.25-1.50%

The following Table 2 shows one embodiment of a formulation for composition made by the method of the present invention.

TABLE 2

| Inqredient | % w/w | Weight (mg) |
|---|---|---|
| Pyrilamine Maleate | 2.91% | 16.00 |
| Phenylephrine HCl | 1.82% | 10.00 |
| Tannic Acid, USP | 8.81% | 48.43 |
| Magnesium Aluminum Silicate, NF | 2.00% | 11.00 |
| Mannitol | 29.24% | 160.82 |
| Methocel E-10M | 1.50% | 8.25 |
| Corn Starch | 1.00% | 5.50 |
| Di-Pac (Sucrose) | 42.00% | 231.00 |
| Calcium Phosphate Dibasic | 3.00% | 16.50 |
| Xanthan Gum | 2.00% | 11.00 |
| Grape Flavor | 1.30% | 7.15 |
| Talc | 0.35% | 1.92 |
| Magnesium Stearate | 0.35% | 1.92 |
| Dextromethorphan Hydrobromide | 2.73% | 15.01 |
| Citric Acid | 0.50% | 2.75 |
| Sucralose | 0.50% | 2.75 |
| Total | 100.00% | 550.000 |

During the manufacturing process, a paddle blender may be used which provides very good mixing of the powder during the conversion step and serves to prevent the formation of the lumps in the formulation. Following addition of excipients, as discussed above, blend samples may be taken during the mixing to show good uniformity of the actives. The granulation exhibits good flow properties and medium oval tablets of 550 mg and 10-12 kp hardness may be manufactured. Each tablet of this embodiment produced by the method of the invention may include 30 mg pyrilamine tannate, 25 mg phenylephrine tannate, and 25 mg dextromethorphan tannate. The composition of this embodiment of the present invention can be used to treat coryza.

The principles of the present method of the invention will be more apparent with reference to the following Examples.

### EXAMPLE 1

### Process of Conversion to Tannate Salts of Phenylephrine, Pyrilamine, and Dextromethorphan

The salt of the active ingredient, corresponding to an amount of free base present in a final batch size of 1 kg was dissolved in 100 ml of purified water. 120 ml of purified water was placed in a 600 ml beaker and stirred. While stirring, 3 g of MAS was added in small portions to obtain a dispersion. The amount of MAS used is a part of the total amount of MAS to be used in the formulation. Once the MAS was dispersed, tannic acid was added to the mixture and stirred to form a uniform dispersion. Three different batches of the MAS/tannic acid dispersion in purified water were prepared for each active. In the three batches, the amount of tannic acid used in the batch may vary from an amount equal to that of the free base, to two time to three times that of the free base present in the initial salt solution.

The salt solution was then added in small portions, under light stirring, to the MAS/Tannic acid dispersion. After all of the salt solution was added, the volume was made up to 250 ml with purified water and stirring was continued for a period of 10 minutes.

The MAS was used in this step to serve as an adherent or a solid support for the tannic acid molecules to facilitate the conversion process. In addition, it also prevented the clumping of the tannate salt formed, which aided in the dispersion of the precipitate of the tannate salt formed from the solution.

The pyrilamine salt solution, upon addition to the MAS/tannic acid dispersion, resulted in the formation of copious amounts of precipitate at all three concentrations of tannic acid. However, in the case of phenylephrine, the tannate salt showed partial solubility in purified water. In the case of dextromethorphan, the tannate salt will result in the formation of copious amounts of precipitate.

The above batches were assayed for the formation of the tannate salt. For pyrilamine and phenylephrine, it was found that maximum conversion (close to 97%) was achieved when tannic acid was used at 3 times the amount of the free base. This conversion rate is expected for dextromethorphan as well.

The foregoing is considered as illustrative only of the principles of the invention. Further, various modifications may be made of the invention without departing form the scope thereof and it is desired, therefore, that only such limitations shall be placed thereon as are imposed by the prior art and which are set forth in the appended claims.

What is claimed is:

1. A composition comprising the active pharmaceutical ingredients phenylephrine, pyrilamine, and dextromethorphan, the composition formed from the steps of:
a. dissolving active pharmaceutical ingredients consisting of phenylephrine, pyrilamine, and dextromethorphan in a first solvent to form a first solution, wherein said active pharmaceutical ingredients are dissolved under conditions that will not cause decomposition of the active pharmaceutical ingredients;
b. mixing a dispersing agent and tannic acid in a second solvent to form a first dispersion;
c. transferring at least a portion of the first solution to the first dispersion, to form a second solution including tannate salts of said active pharmaceutical ingredients;
d. combining substances selected from the group consisting of preservatives, suspending agents, thickening agents, coloring agents, anti-caking agents, sweetening agents, flavoring agents and pH adjusting agents to form a liquid pharmaceutical carrier; and
e. combining at least a portion of the second solution to the liquid pharmaceutical carrier to produce a liquid dosage form including pharmaceutically active tannate salts.

2. The composition of claim 1 wherein the active pharmaceutical ingredients are present in a range of about 0.05% to about 25.0% by weight.

3. The composition of claim 1 wherein the active pharmaceutical ingredients are selected from the group of salts consisting of maleate, citrate, hydrochloride, hydrobromide, acetate, and sulfate.

4. The composition of claim 1 wherein the tannic acid is natural or synthetic.

5. The composition of claim 1 wherein the dispersing agent is selected from the group consisting of magnesium aluminum silicate, xanthan gum and cellulose compounds.

6. The composition of claim 5 wherein the dispersing agent is magnesium aluminum silicate and is present in a range of about 0.05% to about 5.0% by weight.

7. The composition of claim 1 wherein the tannic acid is present in a range of about 0.01 to about 30.0% by weight.

8. The composition of claim 6 wherein the magnesium aluminum silicate and tannic acid are present by weight in a ratio in the range of 0.1:1 to 100:1.

9. The composition of claim 1 wherein the tannic acid and the active pharmaceutical ingredients are present by weight in a ratio in the range of 1:1 to 10:1.

10. The composition of claim 1 wherein the thickening agent is magnesium aluminum silicate and is present in a range of about 0.5% to about 10.0% by weight.

11. The composition of claim 1 wherein the suspending agent is xanthan gum and is present in a range of about 0.5 to about 10.0% by weight.

12. The composition of claim 1 wherein the sweetening agents include sucrose present in a range of about 5.0% to about 50.0% by weight, and sucralose and magnasweet MM-100 present in a range of about 0.01% to about 3.0% by weight.

13. The composition of claim 1 wherein the flavoring agent is artificial grape and is present in a range of about 0.01% to about 2.0% by weight.

14. The composition of claim 1 wherein the second solvent is water and is present in a range of about 10.0 to about 85.0% by weight.

15. The composition of claim 1 wherein said second solvent is glycerin and is present in a range of about 2.5% to about 20.0% by weight.

16. The composition of claim 1 wherein the preservative is methylparaben and is present in a range of about 0.01 to about 1.0% by weight.

17. The composition of claim 1 wherein the pH adjusting agents are sodium benzoate, citric acid, and sodium citrate and are present in a range of about 0.05 to about 1.0% by weight.

18. The composition of claim 1 wherein the anti-caking agent is MAS and is present in the range of about 0.5 to about 10.0% by weight.

19. The composition of claim 1 wherein the pH of said liquid dosage form is in a range of about 3.5 to about 6.5.

20. The composition of claim 1 wherein the pharmaceutically active tannate salts are pyrilamine tannate present at about 30 mg, phenylephrine tannate present at about 12.5 mg, and dextromethorphan tannate present at about 25 mg.

21. The composition of claim 19 wherein said liquid dosage form is a suspension.

22. A manufacturing process for the formation of a combination of pharmaceutically active tannate salts selected from the group consisting of phenylephrine, pyrilamine, and dextromethorphan, which comprises the steps of:
a. dissolving active pharmaceutical ingredients consisting of phenylephrine, pyrilamine, and dextromethorphan in a first solvent to form a first solution, wherein said active pharmaceutical ingredients are dissolved under conditions that will not cause decomposition of the active pharmaceutical ingredients;
b. mixing a dispersing agent and tannic acid in a second solvent to form a first dispersion;
c. transferring at least a portion of the first solution to the first dispersion, to form a second solution including tannate salts of said active pharmaceutical ingredients;
d. forming a liquid pharmaceutical carrier by combining substances selected from the group consisting of preservatives, suspending agents, thickening agents, coloring agents, anti-caking agents, sweetening agents, flavoring agents and pH adjusting agents; and
e. combining at least a portion of the second solution with the liquid pharmaceutical carrier to produce a liquid dosage form including pharmaceutically active tannate salts.

23. The process of claim 22, wherein forming a liquid pharmaceutical carrier further comprises combining suspending agents, thickening agents, coloring agents, sweetening agents, and flavoring agents in a third solvent to form a third solution.

24. The process of claim 23, wherein forming a liquid pharmaceutical carrier further comprises combining preservatives, anti-caking agents, and pH adjusting agents to a fourth solvent to form a second dispersion.

25. The process of claim 24, further comprising transferring at least a portion of the second solution to the third solution to form a third dispersion.

26. The process of claim 25, further comprising transferring at least a portion of the second dispersion to the third dispersion.

27. The process of claim 22, wherein the active pharmaceutical ingredients are provided as salts or in free base form.

28. The process of claim 22 wherein dissolving the active pharmaceutical ingredients in a first solvent occurs at a temperature in range of about 20° C. to about 50° C.

29. The process of claim 22 wherein dissolving an active pharmaceutical ingredients in a first solvent occurs at a pH range of about 3 to about 11.

30. The process of claim 22 wherein the liquid dosage form is for immediate or prolonged release of the active ingredients.

31. A composition comprising active pharmaceutical ingredients selected from the group consisting of phenylephrine, pyrilamine, and dextromethorphan, the composition formed from the steps of:
a. dissolving active pharmaceutical ingredients consisting of phenylephrine, pyrilamine, and dextromethorphan in a first solvent to form a first solution, wherein said active pharmaceutical ingredient are dissolved under conditions that will not cause decomposition of the active pharmaceutical ingredients;
b. mixing a dispersing agent, diluent and tannic acid to form a first powder mixture;
c. transferring at least a portion of the first solution to the first powder mixture, to form tannate salts of said active pharmaceutical ingredients in a granulate;
d. combining the granulate with one or more substances selected from the group consisting of diluents, dry binding/matrix forming agents, binding solutions, coloring agents, sweetening agents, hardness-increasing agents, flavoring agents, and excipients; and
f. processing the granulate into solid dosage forms.

32. The process of claim 31 wherein the active pharmaceutical ingredients are free bases or salts selected form the group consisting of maleate, citrate, chloride, hydrochloride, bromide, hydrobromide, acetate, sulfate, mesylate, palmitate, and stearate.

33. The process of claim 31 wherein the tannic acid is natural or synthetic.

34. The process of claim 31 wherein the dispersing agent is selected from the group consisting of magnesium aluminum silicate, xanthan gum and cellulose compounds.

35. The process of claim 31 wherein the solvents are selected from the group consisting of purified water, ethanol, diethylether, methylene chloride, acetone, and isopropyl alcohol.

36. The process of claim 31 wherein the diluent is selected from the group consisting of lactose, microcrystalline cellulose, sucrose and mannitol and is present in a concentration of about 1.0% to about 75.0%.

37. The process of claim 31 wherein the binder solution comprises material selected from the group consisting of corn starch, pregelatinized starch, potato starch, polyvinylpyrrolidone and xanthan gum and is present in a concentration of about 0.1% to about 20.0%.

38. The process of claim 37 wherein the binder solution further comprises a solvent.

39. The process of claim 38 wherein the solvent is selected from the group consisting of purified water, ethanol, diethylether, methylene chloride, acetone, and isopropyl alcohol

40. The process of claim 31 wherein the dry binding/matrix forming agents are selected from the group consisting of methylcellulose, hydroxypropyl methyl cellulose, ethylcellulose, hydroxypropyl cellulose, xanthan gum and polyvinyl pyrrolidone and each is present at a concentration of about 0.1% to about 20.0%.

41. The process of claim 31 wherein the coloring agents are selected from the group consisting of blue, red, yellow, green, orange, and purple and each is present at a concentration of about 0.01% to about 2.0%.

42. The process of claim 31 wherein the sweetening agents are selected from the group consisting of sucrose, saccharin sodium, xylitol, magnasweet MM-100, and sucralose and each is present at a concentration of about 0.01% to about 40.0%.

43. The process of claim 31 wherein the flavoring agents are selected from grape, cherry, orange, lime and strawberry and is present in a concentration of about 0.01% to about 3.0%.

44. The process of claim 31 wherein the dispersing agent is magnesium aluminum silicate and is present in about 0.05% to about 15.0% by weight.

45. The process of claim 31 wherein the tannic acid is present in the range of about 0.05% to about 30.0% by weight.

46. The process of claim 44 wherein the ratio of magnesium aluminum silicate to tannic acid is present in the weight ratio of 0.1:1 to 100:1.

47. The process of claim 31 wherein the tannic acid and the active pharmaceutical ingredients are present in the weight ratio 1:1 to 10:1.

48. The process of claim 31 wherein the tannate salts are pyrilamine tannate present at 30 mg, phenylephrine tannate present at 25 mg, and dextromethorphan tannate present at 25 mg.

49. A manufacturing process for the formation of a combination of pharmaceutically active tannate salts selected from the group consisting of phenylephrine, pyrilamine, an dextromethorphan, as therapeutic solid dosage form for human use, which comprises the steps of:
a. dissolving active pharmaceutical ingredients consisting of phenylephrine, pyrilamine, and dextromethorphan in a first solvent to form a first solution, wherein said active pharmaceutical ingredient are dissolved under conditions that will not cause decomposition of the active pharmaceutical ingredients;
b. mixing a dispersing agent, diluent and tannic acid to form a first powder mixture;
c. transferring at least a portion of the first solution to the first powder mixture, to form tannate salts of said active pharmaceutical ingredients in a granulate;
d. combining the granulate with one or more substances selected from the group consisting of diluents, dry binding/matrix forming agents, binder solutions, coloring agents, sweetening agents, hardness-increasing agents, flavoring agents, and excipients; and
f. processing the granulate into solid dosage forms.

50. The process of claim 49 wherein when combining excipients with the granulation the excipients are selected from the group consisting of calcium phosphate, calcium stearate, talc, colloidal silica, magnesium stearate and stearic acid and each is present at a concentration of about 0.1% to about 10.0%.

51. The process of claim 49 wherein dissolving the active pharmaceutical ingredient in a first solvent occurs at a temperature in the range of about 20° C. to about 50° C.

52. The process of claim 49 wherein dissolving the active pharmaceutical ingredient in a first solvent occurs at a pH in a range of about 7 to about 11.

53. A composition comprising tannate salts being formed by a method comprising:
a. dissolving active pharmaceutical ingredients selected from the group consisting of phenylephrine, pyrilamine, and dextromethorphan in a first solvent to form a first solution, wherein said active pharmaceutical ingredients are dissolved at a temperature and pH value that will not cause decomposition of the active pharmaceutical ingredients;
b. mixing a dispersing agent and tannic acid in a second solvent to form a first dispersion; and
c. transferring at least a portion of the first solution to the first dispersion, to form a second solution including tannate salts of the active pharmaceutical ingredients.
